SEQUENCE LISTING (72 sequences)

SEQ ID NO: 1 - DNA, 31 bp, Artificial Sequence
  Description of Artificial Sequence Synthetic oligonucleotide
gatccttactagttttagtagcggccgcggg

SEQ ID NO: 2 - DNA, 31 bp, Artificial Sequence
  Description of Artificial Sequence Synthetic oligonucleotide
aattcccgcggccgctactaaaactagtaag

SEQ ID NO: 3 - DNA, 59 bp, Artificial Sequence
  Description of Artificial Sequence Synthetic oligonucleotide
gaccgcgtttgccggaacggcaatcagcatcgttcaccaccaccaccaccactaatagg

SEQ ID NO: 4 - DNA, 60 bp, Artificial Sequence
  Description of Artificial Sequence Synthetic oligonucleotide
aattcctattagtggtggtggtggtggtgaacgatgctgattgccgttccggcaaacgcg

SEQ ID NO: 5 - DNA, 48 bp, Artificial Sequence
  Description of Artificial Sequence Synthetic oligonucleotide
tttatctagacccagccctaggaagcttctcctgagtaggacaaatcc

SEQ ID NO: 6 - DNA, 32 bp, Artificial Sequence
  Description of Artificial Sequence Synthetic oligonucleotide
gggtctagataaaacgaaaggcccagtctttc

SEQ ID NO: 7 - DNA, 56 bp, Artificial Sequence
  Description of Artificial Sequence Synthetic oligonucleotide
ccgccttccatgggtactagttttaaatgcggccgcacgagcaaagaaacctttac

SEQ ID NO: 8 - DNA, 21 bp, Artificial Sequence
  Description of Artificial Sequence Synthetic oligonucleotide
ctctcatccgccaaaacagcc

SEQ ID NO: 9 - DNA, 20 bp, Artificial Sequence
  Description of Artificial Sequence Synthetic oligonucleotide
gcggccgctggnnnnnnnnn

SEQ ID NO: 10 - DNA, 19 bp, Artificial Sequence
  Description of Artificial Sequence Synthetic oligonucleotide
ggcggccaacnnnnnnnnn

SEQ ID NO: 11 - DNA, 19 bp, Artificial Sequence
  Description of Artificial Sequence Synthetic oligonucleotide
gcactagtggccggccaac

SEQ ID NO: 12 - DNA, 20 bp, Artificial Sequence
  Description of Artificial Sequence Synthetic oligonucleotide
gcactagtcggccggccaac

SEQ ID NO: 13 - DNA, 21 bp, Artificial Sequence
  Description of Artificial Sequence Synthetic oligonucleotide
gcactagtcgggccggccaac

SEQ ID NO: 14 - DNA, 21 bp, Artificial Sequence
  Description of Artificial Sequence Synthetic oligonucleotide
ggaggctcgagcggccgctgg

SEQ ID NO: 15 - DNA, 188 bp, Homo sapiens
atgggtactagtcggccggccaacatcactcccaccaatacaatgacttctatgagaactacaacctattggcccacagccacaatgatggaaccaccttcatccactgtatcaactacaggcagaggtcagaccacctttccagctctacagccacattccccaataccaaacaccccagcggccgc

SEQ ID NO: 16 - DNA, 150 bp, Homo sapiens
atgggtactagtcgggccggccaacttgttgaagaactggataaagtggaatctcaagaacgagaagatgttctggctggaatgtctggaaaatcctctttccaaagatctgaaggagattttcttttaagatcattgaccagcggccgc

SEQ ID NO: 17 - DNA, 189 bp, Homo sapiens
atgggtactagtggccggccaacaaggcagctggaagaggttctcaaattagatcaagaaatgcctttaacagaagtgaagagtgaacctgaggaaaatatcgattcaaacagtgaaagtgaaagagaagagatagaattaaaatctccgaggggacgaaggagaattgctcgagatcccagcggccgc

SEQ ID NO: 18 - DNA, 312 bp, Homo sapiens
atgggtactagtcgggccggccaacttgaggagctgcagaagaaataccagcaaaagctagagcaggaggagaaccctggcaatgataatgtaacaattatggagctacagacacagctagcacagaagacgactttaatcagtgattcgaaattgaaagagcaagagttcagagaacagattcacaatttagaagaccgtttgaagaaatatgaaaagaatgtatatgcaacaactgtggggacaccttacaaaggtggcaatttgtaccatacggatgtctcactctttggagaacctaccagcggccgc

SEQ ID NO: 19 - DNA, 165 bp, Homo sapiens
atgggtactagtcggccggccaacttcgtggaaatcagtgaagataaaactaaaatcagaaggtctccaagcaaacccctacctgaagtgactgatgagtataaaaatgatgtaaaaaacagatctgtttatattaaaggcttcccaactgaagccagcggccgc

SEQ ID NO: 20 - DNA, 132 bp, Homo sapiens
gtggccggccaacgttatcagagtagaagtgggcatgatcagaagaatcatagaaagcatcatgggaagaaaagaatgaaaagtaaacgatctacatcattgtcatctcccagaaacggaaccagcggccgc

SEQ ID NO: 21 - DNA, 189 bp, Homo sapiens
atgggtactagtcgggccggccaacaaattaggcagattgagtgtgacagtgaagacatgaagatgagagctaagcagctcctggttgcctggcaagatcaagagggagttcatgcaacacctgagaatctgattaatgcactgaataagtctggattaagtgaccttgcagaaagtcccagcggccgc

SEQ ID NO: 22 - DNA, 180 bp, Homo sapiens
atgggtactagtggccggccaacggcagtagttctggaaaagccactggggacgagacaggtgctaaagttgaacgagctgatggagcttcatggtgaaggcagtagttctggaaaagccactggggacgagacaggtgctaaagttgaacgagctgatggaatgacccccagcggccgc

SEQ ID NO: 23 - DNA, 160 bp, Homo sapiens
atgggtactagtggccggccaacgaattattcgagtgctataggcgcttgtcagggaggtagcgatgagagtaatagatagggctcaggcgtttgttgatgagatatttggaggtggggatgatgcacataatttgaatcaacacaactccagcggccgc

SEQ ID NO: 24 - DNA, 162 bp, Homo sapiens
atgggtactagtcgggccggccaacgtggtattatttaaaaatagctaaaaaggtaaacaatccaaatgccattaaacagagaattttaaaaaatgagatactacacagcaacaaaaacctatgagctaatgctagatgcaacaacacagaccagcggccgc

SEQ ID NO: 25 - DNA, 81 bp, Homo sapiens
atgggtactagtcgggccggccaactacacgcctttccactccactctactacactctactacactacacccagcggccgc

SEQ ID NO: 26 - DNA, 87 bp, Homo sapiens
atgggtactagtcggccggccaacagagaagctaagcaactgcatcatcagccacattcaatcgaattaatacagtccagcggccgc

SEQ ID NO: 27 - DNA, 66 bp, Homo sapiens
atgggtactagtcggccggccaactcagaggtgtataagccaacattgctctactccagcggccgc

SEQ ID NO: 28 - DNA, 69 bp, Homo sapiens
atgggtactagtggccggccaacggttggttttactctagatttcactgtcgacccacccagcggccgc

SEQ ID NO: 29 - DNA, 60 bp, Homo sapiens
atgggtactagtcgggccggccaactataccgtacaaccctaacatataccagcggccgc

SEQ ID NO: 30 - DNA, 282 bp, Homo sapiens
atgggtactagtcgggccggccaacagagagagcaagaaaagaaaagaagccctcaagatgttgaagttctcaagacaactactgagctatttcatagcaatgaagaaagtggattttttaatgaactcgaggctcttagagctgaatcagtggctaccaaagcagaacttgccagttataaagaaaaggctgaaaaacttcaagaagaacttttggtaaaagaaacaaatatgacatctcttcagaaagacttaagccaagttagggatcaccagggccgc

SEQ ID NO: 31 - DNA, 435 bp, Homo sapiens
  modified_base (297): a, c, g, t, unknown, or other
atgggtactagtcgggccggccaacacgcattcgagcaaataccaagtcgccagaagaaaattttagaagaagctcatgaattgagtgaagatcactataagaaatatttggcaaaactcaggtctattaatccaccatgtgtgcctttctttggaatttatctcactaatctcttgaaaacagaagaaggcaaccctgaggtcctaaaaagacatggaaaagagcttataaactttagcaaaaggaggaaagtagcagaaataacaggagagatccagcagtaccaaaatcagccntactgtttacgagtagaatcagatatcaaaaggttctttgaaaacttgaatccgatgggaaatagcatggagaaggaatttacagattatcttttcaacaaatccctagaaatagaaccacgaaaacccagcggccgc

SEQ ID NO: 32 - DNA, 331 bp, Homo sapiens
atgggtactagtcgggccggccaacaggagaggtccttggccctctgtgaaccaggtgtcaatcccgaggaacaactgattataatccaaagtcgtctggatcagagtttggaggagaatcaggacttaaagaaggaactgctgaaatgtaaacaagaagccagaaacttacaggggataaaggatgccttgcagcagagattgactcagcaggacacatctgttcttcagctcaaacaagagctactgagggcaaatatggacaaagatgagctgcacaaccagaatgtggatctgcagaggaagctagatgagaggacccagcggccgc

SEQ ID NO: 33 - DNA, 201 bp, Homo sapiens
atgggtactagtcgggccggccaaccgatgtctggacatgggagttttcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccaacccagcggccgc

SEQ ID NO: 34 - DNA, 219 bp, Homo sapiens
actagtcgggccggccaacgttatgagaagtcagatagtagcgatagtgagtatatcagtgatgatgagcagaagtctaagaacgagccagaagacacagaggacaaagaaggttgtcagatggacaaagagccatctgctgttaaaaaaaagcccaagcctacaaacccagtggagattaaagaggagcttaaaagcacgccaccagccagcggccgc

SEQ ID NO: 35 - DNA, 72 bp, Homo sapiens
actagtcgggccggccaacttgccaggattccctcagtaacggcgagtgaacagggaagaaccagcggccgc

SEQ ID NO: 36 - DNA, 152 bp, Homo sapiens
actagtgggccggccaacgctgctccaccctcagcagatgataatatcaagacacctgccgagcgtctgcgggggccgcttccaccctcagcggatgataatctcaagacaccttccgagcgtcagctcactcccctccccccagcggccgc

SEQ ID NO: 37 - DNA, 423 bp, Homo sapiens
actagtcgggccggccaacgggaattgggaaggacgggcctatatccctcctacaaagttcgagagaagatagaaacggtcaagtaccccacatatcctgaggctgagaaataaagctcagatggaagagataaacgaccaaactcagttcgaccaaactcagttcaaaccatttgagccaaactgtagatgaagagggctctgatctaacaaaataaggttatatgagtagatactctcagcaccaagagcagctgggaactgacataggcttcaattggtggaattcctctttaacaagggctgcaatgccctcatacccatgcacagtacaataatgtactcacatataacatgcaaaggttgttttctactttgcccctttcagtatgtccccataagacaaacactaccagcggccgc

SEQ ID NO: 38 - DNA, 237 bp, Homo sapiens
actagtgtcctggaacccacaaaagtaaccttttctgtttcaccgattgaagcgacggagaaatgtaagaaagtggagaagggtaatcgagggcttaaaaacataccagactcgaaggaggcacctgtgaacctgtgtaaacctagtttaggaaaatcaacaatcaaaacgaataccccaataggctgcaaagttagaaaaactgaaattataagttacccaagtaccagcggccgc

SEQ ID NO: 39 - DNA, 228 bp, Homo sapiens
atggacttaacagctgtttacagaacattccacccaacaatcacagaatatacattctatttaacagtgcatggaactttttccaagatagaccatatgataggccacaaaacaagtctcaataagtctaagaaaactgaaattatatcaagtactctctcagaccacagtggaataaaattggaaagtaattccaaaaggaacccccaaatccatgccagcggccgc

SEQ ID NO: 40 - DNA, 189 bp, Homo sapiens
atgccgattgacgttgtttacacctgggtgaatggcacagatcttgaactactgaaggaactacagcaggtcagagaacagatggaggaggagcagaaagcaatgagagaaatccttgggaaaaacacaacggaacctactaagaagaggtcctactttgtgaattttctagccgtgtccagcggccgc

SEQ ID NO: 41 - DNA, 318 bp, Homo sapiens
actagtggccggccaacgtataaagtaaatatttctaaagcaaaaactgctgtgacggagctcccttctgcaaggacagatacaacaccagttataaccagtgtgatgtcattggcaaaaatacctgctaccttatctacagggaacactaacagtgttttaaaaggtgcagttactaaagaggcagcaaagatcattcaagatgaaagtacacaggaagatgctatgaaatttccatcttcccaatcttcccagccttccaggcttttaaagaacaaaggcatatcatgcaaacccgtcacacatcccagcggccgc

SEQ ID NO: 42 - DNA, 273 bp, Homo sapiens
actagtcgggccggccaacttcgatttagtgatcatgccgtgttgaaatccttgtctcctgtagacccagtggaacccataagtaattcagaaccatcaatgaattcagatatgggaaaagtcagtaaaaatgatactgaagaggaaagtaataaatccgccacaacagacaatgaaataagtaggactgagtatttatgtgaaaactctctagaaggtaaaaataaagataattcttcaaatgaagtcttcccccaatatgccagcggccgc

SEQ ID NO: 43 - DNA, 258 bp, Homo sapiens
actagtcgggccggccaacgcaagcaaagtttcccaaattcagatcctttacatcagtctgatacttccaaagctccaggttttagaccaccattacagagacctgctccaagtccctcaggtattgtcaatatggactcgccatatggttctgtaacaccttcttcaacacatttgggaaactttgcttcaaacatttcaggaggtcagatgtacggacctggggcaccccttggaggagcacccaccagcggccgc

SEQ ID NO: 44 - DNA, 138 bp, Homo sapiens
atgggtactagtcgggccggccaacccacttcagaaaactatttggcagtaactactaaaactaaacataagcatagcctacaacccagtaatgccagtatttcactcctaggtatatacccaacccccagcggccgc

SEQ ID NO: 45 - DNA, 99 bp, Homo sapiens
actagtcgggccggccaacgtgacacacagacacatgcacatgtgagtgtatgcgtgcacacaccccaccacacctacaaataccccaccagcggccgc

SEQ ID NO: 46 - protein, 46 aa, Homo sapiens
VLVAGQRYQSRSGHDQKNHRKHHGKKRMKSKRSTSLSSPRNGTSGR

SEQ ID NO: 47 - protein, 50 aa, Homo sapiens
MGTSRAGQLVEELDKVESQEREDVLAGMSGKSSFQRSEGDFLLRSLTSGR

SEQ ID NO: 48 - protein, 27 aa, Homo sapiens
MGTSRAGQLHAFPLHSTTLYYTTPSGR

SEQ ID NO: 49 - protein, 29 aa, Homo sapiens
MGTSRPANREAKQLHHQPHSIELIQSSGR

SEQ ID NO: 50 - protein, 22 aa, Homo sapiens
MGTSRPANSEVYKPTLLYSSGR

SEQ ID NO: 51 - protein, 23 aa, Homo sapiens
MGTSGRPTVGFTLDFTVDPPSGR

SEQ ID NO: 52 - protein, 20 aa, Homo sapiens
MGTSRAGQLYRTTLTYTSGR

SEQ ID NO: 53 - protein, 23 aa, Homo sapiens
MRYYTATKTYELMLDATTQTSGR

SEQ ID NO: 54 - protein, 32 aa, Homo sapiens
MRVIDRAQAFVDEIFGGGDDAHNLNQHNSSGR

SEQ ID NO: 55 - protein, 95 aa, Homo sapiens [sequence omitted]

SEQ ID NO: 56 - protein, 144 aa, Homo sapiens
AGTSRAGQHAFEQIPSRQKKILEEAHELSEDHYKKYLAKLRSINPPCVPFFGIYLTNLLKTEEGNPEVLKRHGKELINFSKRRKVAEITGEIQQYQNQYCLRVESDIKRFFENLNPMGNSMEKEFTDYLFNKSLEIEPRKPSGR

SEQ ID NO: 57 - protein, 110 aa, Homo sapiens
MGTSRAGQQERSLALCEPGVNPEEQLIIIQSRLDQSLEENQDLKKELLKCKQEARNLQGIKDALQQRLTQQDTSVLQLKQELLRANMDKDELHNQNVDLQRKLDERTQRP

SEQ ID NO: 58 - protein, 67 aa, Homo sapiens [sequence omitted]

SEQ ID NO: 59 - protein, 70 aa, Homo sapiens
TSRAGQRYEKSDSSDSEYISDDEQKSKNEPEDTEDKEGCQMDKEPSAVKKKPKPTNPVEIKEELKSTPPA

SEQ ID NO: 60 - protein, 21 aa, Homo sapiens
TSRAGQLARIPSVTASEQGRT

SEQ ID NO: 61 - protein, 52 aa, Homo sapiens
TSGPANAAPPSADDNIKTPAERLRGPLPPSADDNLKTPSERQLTPLPPAAAK

SEQ ID NO: 62 - protein, 37 aa, Homo sapiens
TSRAGQRELGRTGLYPSYKVREKIETVKYPTYPEAEK

SEQ ID NO: 63 - protein, 79 aa, Homo sapiens
TSVLEPTKVTFSVSPIEATEKCKKVEKGNRGLKNIPDSKEAPVNLCKPSLGKSTIKTNTPIGCKVRKTEIISYPSTSGR

SEQ ID NO: 64 - protein, 76 aa, Homo sapiens
MDLTAVYRTFHPTITEYTFYLTVHGTFSKIDHMIGHKTSLNKSKKTEIISSTLSDHSGIKLESNSKRNPQIHASGR

SEQ ID NO: 65 - protein, 63 aa, Homo sapiens
MPIDVVYTWVNGTDLELLKELQQVREQMEEEQKAMREILGKNTTEPTKKRSYFVNFLAVSSGR

SEQ ID NO: 66 - protein, 106 aa, Homo sapiens
TSGRPTYKVNISKAKTAVTELPSARTDTTPVITSVMSLAKIPATLSTGNTNSVLKGAVTKEAAKIIQDESTQEDAMKFPSSQSSQPSRLLKNKGISCKPVTHPSGR

SEQ ID NO: 67 - protein, 91 aa, Homo sapiens
TSRAGQLRFSDHAVLKSLSPVDPVEPISNSEPSMNSDMGKVSKNDTEEESNKSATTDNEISRTEYLCENSLEGKNKDNSSNEVFPQYASGR

SEQ ID NO: 68 - protein, 86 aa, Homo sapiens
TSRAGQRKQSFPNSDPLHQSDTSKAPGFRPPLQRPAPSPSGIVNMDSPYGSVTPSSTHLGNFASNISGGQMYGPGAPLGGAPTSGR

SEQ ID NO: 69 - protein, 46 aa, Homo sapiens [sequence omitted]

SEQ ID NO: 70 - protein, 33 aa, Homo sapiens
TSRAGQRDTQTHAHVSVCVHTPHHTYKYPTSGR

SEQ ID NO: 71 - DNA, 24 bp, Artificial Sequence
  Description of Artificial Sequence Synthetic oligonucleotide
cgattaaataaggaggaataaacc

SEQ ID NO: 72 - DNA, 21 bp, Artificial Sequence
  Description of Artificial Sequence Synthetic oligonucleotide
ctctcatccgccaaaacagcc

(19) United States
(12) Patent Application Publication
(10) Pub. No.: US 2005/0084857 A1
(43) Pub. Date: Apr. 21, 2005

(54) Identification of specific tumour antigens by means of the selection of cdna libraries with sera and the use of said antigens in diagnostic imaging techniques

(75) Inventors: Franco Felici, Rome (IT); Olga Minenkova, Rome (IT)

Correspondence Address: NIXON & VANDERHYE, PC, 1100 N GLEBE ROAD, 8TH FLOOR, ARLINGTON, VA, 22201-4714, US

(73) Assignee: Kenton S.R.L., Pomezia(Rome) (IT)

(21) Appl. No.: 10/484,917

(22) PCT Filed: Jul. 25, 2002
(86) PCT No.: PCT/IT02/00491

(30) Foreign Application Priority Data
Jul. 26, 2001 (IT) 01/00405

(51) Int. Cl. C12Q 1/68; G01N 33/574; C07K 14/47

(52) U.S. Cl. 435/6; 435/7.23; 435/69.3; 435/320.1; 435/325; 530/324; 530/326

(57) ABSTRACT

A method is described for the identification of specific tumor antigens by means of the selection of cDNA display libraries by using sera, characterised in that said selection is accomplished with the phage display technique, and in particular said selection is accomplished by means of the SEREX technique (serological analysis of autologous tumor antigens through the expression of recombinant cDNA). The method according to the invention described herein advantageously combines the SEREX approach with the potency of the phage display technique defined above, at the same time avoiding the drawbacks characteristic of the SEREX technique. The so identified antigens are useful for the preparation of medicaments for the treatment of tumors.

The invention described herein relates to a method for the identification of specific tumour antigens by means of selection with sera of cDNA libraries derived from subjects suffering from tumours, and particularly for the diagnosis of tumours.

The invention described herein also relates to the technical field of the preparation of diagnostic aids not used directly on the animal or human body.

The invention described herein provides compounds, methods for their preparation, methods for their use, and compositions containing them, suitable for industrial application in the pharmaceutical field.

The invention described herein provides compounds, compositions and methods suitable for substances useful in diagnostic medicine, such as in imaging techniques for the detection and diagnosis of pathological abnormalities of organs and tissues.

In particular, though not exclusively so, the invention described herein relates to the tumour diagnostics sector.

### BACKGROUND TO THE INVENTION

Early diagnosis is an important priority and a highly desired objective in all fields of medicine, particularly because it enables an appreciable improvement in the patient's quality of life to be achieved as well as a concomitant saving of expenditure on the part of national health systems and the patients themselves.

Among the various diagnostic techniques available, there is a tendency today to prefer the so-called non-invasive techniques, and, among these, the various imaging techniques, which represent ways of ascertaining the presence of possible pathological abnormalities without subjecting the patient to complex and sometimes painful or dangerous diagnostic investigations, such as those involving taking samples and biopsies.

Among the most commonly used imaging techniques, we may mention computerised tomography (TC), magnetic resonance (MR) ultrasonography (US) and scintigraphy (SC).

These image acquisition techniques require the use of increasingly efficient contrast media. Their development, however, is aimed solely at improving the anatomical characterisation afforded by the images through enhanced sensitivity, without to date succeeding in developing the specificity of the signal for tissue characterisation. Though it is possible today to visualise anatomical lesions even of extremely small size, the definition of the nature of the lesions observed still requires invasive-type investigations.

One solution to this problem is the development of contrast media capable of selectively and specifically increasing the degree of contrast in the image between healthy tissue and pathological lesions.

One example provided by known technology is the use of monoclonal antibodies as the vehicles of contrast agents and attempts in this sense have been made in the fields of SC and MR. Whereas positive results have been achieved with SC techniques, which, however, still require further improvements, the results in MR are as yet unsatisfactory. A similar need to improve the results is also perceived in the field of US.

The identification of tumour antigens may provide new and better reagents for the construction of target-specific contrast media (TSCM). More or less specific tumour antigens are known, which have been obtained using tumour cells as antigens-immunogens to stimulate antibodies in laboratory animals. Also known are a number of tumour antigens that stimulate the formation of antibodies in the patients themselves (for example, p53, HER-2/neu). These types of antigens are in principle excellent candidates as markers discriminating between healthy and tumour tissue. Their identification, however, is difficult when using conventional methods.

The recent development of a method of analysing (screening) cDNA libraries with sera of patients suffering from various types of tumours, known as SEREX (serological analysis of autologous tumour antigens through the expression of recombinant cDNA, see P.N.A.S. 92, 11810-1995), has led to the identification of a large number of tumour antigens.

The SEREX technology is undoubtedly useful for identifying new tumour antigens, but it presents a number of drawbacks consisting in the very laborious nature of the library screening operations, the high degree of background noise and the large amounts of material necessary.

Since 1993, the year the first tumour antigen (carbonic anhydrase) was characterised, more than 600 different proteins specifically expressed in tumours and to which an immune response is generated have been identified (M. Pfreundschuch et al. Cancer Vaccine Week, International Symposium, Oct. 5-9, 1998, S03) and this number is destined to rise still further [as today SEREX database contains 1695 public sequences (www.licr.org/SEREX.html)]. It is interesting to note that 20-30% of the sequences isolated are as yet unknown gene products.

Further research, however, is necessary to improve the techniques for identifying specific tumour antigens for the diagnosis and treatment of tumours.

Abstract of the Invention

It has now been found that a combination of the SEREX technique and phage display, a strategy based on the selection of libraries in which small protein domains are displayed on the surface of bacteriophages, within which the corresponding genetic information is contained, provides a method for the identification of specific tumour antigens by means of the selection of cDNA display libraries with sera. Using this method it proves possible to identify antigens from very large libraries (i.e. which express a large number of different sequences). The antigens thus identified make it possible to be used in the preparation of contrast media or to obtain specific ligands, which in turn can be used in the preparation of contrast media.

Therefore, one object of the invention described herein is a method for the identification of specific tumour antigens by means of the selection of cDNA display libraries with sera, characterised in that said selection is accomplished using the phage display technique.

The purpose of the invention described herein is to provide a method for identifying tumour antigens useful for the preparation of contrast media for the diagnostic imaging of tumour lesions, as well as the contrast media so obtained.

The contrast media can be prepared according to normal procedures well-kown in this field and need no further explanation.

### DETAILED DESCRIPTION OF THE INVENTION

The invention described herein comprises the construction of cDNA libraries from tumour cells, obtained both from biopsies (preferable fresh) and from cultured tumour lines, the selection (screening) of such libraries with autologous and heterologous patient sera to identify tumour antigens, including new ones, the characterisation of said antigens, the generation of specific ligands for said tumour antigens (for example, antobodies, such as recombinant human antibodies or humanised recombinant murine antibodies), and the construction of target-selective contrast media incorporating the ligands generated.

The method, according to the invention described herein, advantageously combines the SEREX approach with the potency of the phage-display technique defined above, at the same time avoiding the drawbacks characteristic of the SEREX technique, as outlined above.

What is meant by “phage display” is, as understood by the person of ordinary skill in the art, a strategy based on the selection of libraries in which small protein domains are exposed on the surface of bacteriophages within which is contained the corresponding genetic information.

The method implemented according to the invention described herein provides for the first time new and advantageous analysis possibilities:
the use of smaller amounts of serum to identify tumour antigens, selecting, prior to screening, the library with sera of patients suffering from tumours, in such a way as to reduce their complexity, enriching it with those clones that express specific antigens;
owing to technical problems, the direct screening of cDNA libraries, as realised with the state of the art technique, does not allow analysis of a large number of clones (more than approximately one million clones), and thus makes it unsuitable to exploit all the potential of recombinant DNA technology. With the method according to the invention, it is, in fact, possible to construct and analyse libraries 10-100 times larger than those traditionally used in SEREX, thus increasing the likelihood of identifying even those antigens which are present to only a limited extent;
lastly, the possibility of effecting subsequent selection cycles using sera of different patients or mixtures of sera facilitates the identification of cross-reactive tumour antigens, which constitute one of the main objectives of the invention described herein.

In a library of cDNA cloned in a non-directional manner, it is expected that approximately one-sixth (16.7%) of the proteins produced will be correct. The enrichment of this type of library with the true translation product is the real task of expression/display libraries. The invention described herein also provides a new vector for the expression of cDNA and the display of proteins as fusions with the amino-terminal portion of bacteriophage lambda protein D (pD) with limited expression of “out-of-frame” proteins. According to the vector design, the phage displays the protein fragment on the surface only if its ORF (“Open Reading Frame”) coincides with that of pD. The average size of the fragments of cloned DNA in our libraries is 100-600 b.p. (base pairs), and for statistical reasons, most of the “out-of-frame” sequences contain stop codons that do not allow translation of pD and display on the phage surface. In this case, the copy of the lambda genome of wild-type gpD supports the assembly of the capsid. The new expression/display vector (λKM4) for cDNA libraries differs from the one used in SEREX experiments (λgt11) in that the recombinant protein coded for by the cDNA fragment is expressed as a fusion with a protein of the bacteriophage itself and thus is displayed on the capsid.

For each library, messenger RNA of an adequate number of cells, e.g. 10⁷ cells, is purified, using common commercially available means, from which the corresponding cDNA has been generated. The latter is then cloned in the expression/display vector λKM4. The amplification of the libraries is accomplished by means of normal techniques known to the expert in the field, e.g. by plating, growth, elution, purification and concentration.

The libraries are then used to develop the conditions required for the selection, “screening” and characterisation of the sequences identified.

A library of the phage-display type, constructed using cDNA deriving from human cells, allows the exploitation of selection by affinity, which is based on the incubation of specific sera with collections of bacteriophages that express portions of human proteins (generally expressed in tumours) on their capsid and that contain within them the corresponding genetic information. Bacteriophages that specifically bind the antibodies present in the serum are easily recovered, in that they remain bound (by the antibodies themselves) to a solid support; the non-specific ones, on the other hand, are washed away.

The “screening”, i.e. the direct analysis of the ability of the single phage clones to bind the antibodies of a given serum, is done only at a later stage, when the complexity of the library (i.e. the different number of sequences) is substantially reduced, as a result of the selection.

The use of selection strategies allows faster analysis of a large number of different protein sequences for the purposes of identifying those that respond to a particular characteristic, for example, interacting specifically with antibodies present in the sera of patients with tumours.

Selection by affinity is based on the incubation of specific sera with collections of bacteriophages that express portions of human proteins (generally expressed in tumours) on their capsid and that contain within them the corresponding genetic information. The bacteriophages that specifically bind antibodies present in the serum are easily recovered in that they remain bound (by the antibodies themselves) to a solid support; the non-specific ones, on the other hand, are washed away.

The “screening”, i.e. the direct analysis of the ability of the single phage clones to bind the antibodies of a given serum, is done only at a later stage, when the complexity of the library (i.e. the different number of sequences) is substantially reduced, as a result of the selection.

This makes it possible to reduce the work burden and, above all, to use a lower amount of serum for each analysis.

The direct “screening” of a classic cDNA library, in fact, entails the use of large amounts of serum, which are not always easy to procure. To analyse a library of approximately 10⁶ independent clones, one would have to incubate with the preselected (autologous) serum the numerous filters containing a total of at least 10⁶ phage plaques transferred from the various Petri dishes with the infected bacteria. Analysing the same library with another serum is possible only when using the amplified library, which means analysing 10⁶ clones, losing the complexity of the original library, or extending the screening 10- to 100-fold and testing 10⁷-10⁸ clones.

This strategy, moreover, does not allow the identification of antigens which are present in only slight amounts in the library or are recognised by antibodies present in low concentrations and does not allow the execution of multiple analyses with different sera.

The use of a library of the phage-display type, on the other hand, allows selection by affinity in small volumes (0.1-1 ml) prior to direct screening, starting from a total of 10¹⁰-10¹¹ phage particles of the amplified library and from limited amounts of serum, such as, for instance, 10 μl. Thus, one can conveniently operate with a library with a complexity 10- to 100-fold greater than the classic library, consequently increasing the probability of identifying those antigens regarded as difficult. For example, when performing two selection cycles and one screening on 82 mm filters, the total overall consumption of serum may be only 40 μl.

Moreover, it is important to note that analysis of a library of the phage-display type may be potentially accomplished with a large number of different sera. It is thus possible to use selection strategies that favour the identification of antigens capable of interacting with the antibodies present in sera of different patients affected by the same type of tumour (cross-reactive antigens).

Various protocols can be adopted based on the use of different solid supports. These protocols are known to experts in the field.

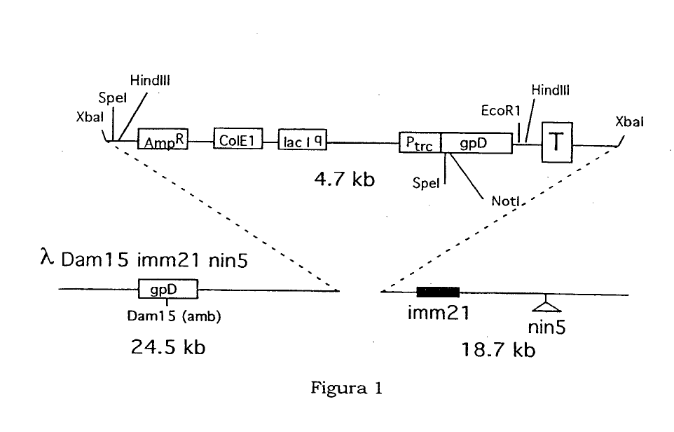

Various protocols can be used based on the use of different solid supports, such as, for example:
sepharose: the serum antibodies with the bound phages are attached to a sepharose resin coated with protein A which specifically recognises the immunoglobulins. This resin can be washed by means of brief centrifuging operations to eliminate the aspecific component;
magnetic beads: the serum antibodies with the bound phages are recovered using magnetic beads coated with human anti-IgC polyclonal antibodies. These beads are washed, attaching them to the test tube wall with a magnet;
Petri dishes: the serum antibodies with the bound phages are attached to a Petri dish previously coated with protein A. The dish is washed by simply aspirating the washing solution.
The invention will now be illustrated in greater detail by means of examples and figures, FIG. 1 representing the map of vector λKM4.

### EXAMPLE

Phages and Plasmids:

Plasmid pGEX-SN was constructed by cloning the DNA fragment deriving from the hybridisation of the synthetic oligonucleotides K108 5′-GATCCTTACTAGTTTTAGTAGCGGCCGCGGG-3′ and K109 5′-AATTCCCGCGGCCGCTACTAAAACTAGTAAG-3′ in the BamHI and EcoRI sites of plasmid pGEX-3X (Smith D. B. and Johnson K. S. Gene, 67(1988) 31-40).

Plasmid pKM4-6H was constructed by cloning the DNA fragment deriving from the hybridisation of the synthetic oligonucleotides K106 5′-GACCGCGTTTGCCGGAACGGCAATCAGCATCGTTCACCACCACCACCACCACTAATAGG-3′ and K107 5′-AATTCCTATTAGTGGTGGTGGTGGTGGTGAACGATGCTGATTGCCGTTCCGGCAAACGCG-3′ in the RsrII and EcoRI sites of plasmid pKM4.

Selection by Affinity

Falcon plates (6 cm, Falcon 1007) were coated for one night at 4° C. with 3 ml of 1 μg/ml of protein A (Pierce, #21184) in NaHCO₃ 50 mM, pH 9.6. After discarding the coating solution, the plates were incubated with 10 ml of blocking solution (5% dry skimmed milk in PBS×1, 0.05% Tween 20) for 2 hours at 37° C. 10 μl of human serum were preincubated for 30 minutes at 37° C. under gentle agitation with 10 μl of BB4 bacterial extract, and 10 μl of MgSO₄ 1M in 1 ml of blocking solution. Approximately 1010 phage particles of the library were added to the serum solution for a further 1 hour incubation at 37° C. under gentle agitation. The incubation mixtures were plated on plates coated with protein A and left for 30 minutes at room temperature. The plates were rinsed several times with 10 ml of washing solution (1×PBS, 1% Triton, 10 mM MgSO₄). The bound phages were recovered by infection of BB4 cells added directly to the plate (600 μl per plate). 10 ml of molten NZY-Top Agar (48-50° C.) were added to the infected cells and immediately poured onto NZY plates (15 cm). The next day, the phages were collected by incubating the plates with agitation with 15 ml of SM buffer for 4 hours at 4° C. The phages were purified by PEG and NaCl precipitation and stored in one tenth of the initial volume of SM with 0.05% sodium azide at 4° C.

Immunoscreening

The phage plaques of the bacterial medium were transferred onto dry nitrocellulose filters (Schleicher & Schuell) for 1 hour at 4° C. The filters were blocked for 1 hour at room temperature in blocking buffer (5% dry skimmed milk in PBS×1, 0.05% Tween 20). 20 μl of human serum were preincubated with 20 μl of BB4 bacterial extract, 10⁹/ml of wild-type lambda phage in 4 ml of blocking buffer. After discarding the blocking solution, the filters were incubated with serum solution for 2 hours at room temperature with agitation. The filters were washed several times with PBS×1, 0.05% Tween 20 and incubated with human anti-IgG secondary antibodies conjugated with alkaline phosphatase (Sigma A 2064) diluted 1:5000. Then the filters were washed as above, rinsed briefly with substrate buffer (100 mM Tris-HCl, pH 9.6, 100 mM NaCl, 5 mM MgCl₂). Each filter was incubated with 10 ml of substrate buffer containing 330 mg/ml nitro blue tetrazolium, 165 mg/ml 5-bromo-4-chloro-3-indolylphosphate. Reaction was stopped by water washing.

Preparation of Lambda Phage on Large Scale (from Lysogenic Cells)

The BB4 cells were grown up to OD₆₀₀=1.0 in LB containing maltose 0.2% with agitation, recovered by centrifugation and resuspended in SM buffer up to OD₆₀₀=0.2. 100 μl of cells were infected with lambda with a low multiplicity of infection, incubated for 20 minutes at room temperature, plated on LB agar with ampicillin and incubated for 18-20 hours at 32° C. The next day, a single colony was incubated in 10 ml of LB with ampicillin for one night at 32° C. with agitation. 500 ml of fresh LB with ampicillin and MgSO₄ 10 mM were inoculated with 5 ml of the overnight culture in a large flask and grown at 32° C. up to OD₆₀₀=0.6 with vigorous agitation. The flask was incubated for 15 minutes in a water bath at 45° C., then incubated at 37° C. in a shaker for a further 3 hours. 10 ml of chloroform were added to the culture to complete the cell lysis and the mixture was incubated in the shaker for another 15 minutes at 37° C. The phage was purified from the lysate culture according to standard procedures (Sambrook, J., Fritsch, E. F & Maniatis, T. (1989) Molecular Cloning, Cold Spring Harbor Laboratory Press, Cold Spring Harbor).

The phage lysates for ELISA were prepared from the lysogenic cells by means of a similar procedure, but without the addition of chloroform. After precipitation with NaCl and PEG, the bacteriophage pellet was resuspended in one tenth of the starting volume of SM buffer with sodium azide (0.05%) and stored at 4° C.

Lambda ELISA

Multi-well plates (Immunoplate Maxisorb, Nunc) were coated for one night at 4° C. with 100 μl/well of anti-lambda polyclonal antibodies at a 0.7 μg/ml concentration in NaHCO₃ 50 mM, pH 9.6. After discarding the coating solution, the plates were incubated with 250 μl of blocking solution (5% dry skimmed milk in PBS×1, 0.05% Tween 20). The plates were washed twice with washing buffer (PBS×1, Tween 20). A mixture of 100 μl of blocking buffer and phage lysate (1:1) was added to each well and incubated for 1 hour at 37° C. 1 ml of human serum was incubated for 30 minutes at room temperature with 10⁹ plaque forming units (pfu) of phage λKM4, 1 μl of rabbit serum, 1 μl of BB4 extract, 1 μl of FBS in 100 μl of blocking buffer. The plates were washed after incubation with phage lysate and incubated with serum solution for 60 minutes at 37° C. The plates were then washed and goat anti-human HRP conjugated antibody was added (Jackson ImmunoResearch Laboratories), at a dilution of 1:20000, in a blocking buffer/secondary antibody mixture (1:40 rabbit serum in blocking solution). After a 30 minute incubation, the plates were washed and peroxidase activity was measured with 100 μl of TMB liquid substrate system (Sigma). After 15 minutes development, the reaction was stopped with 25 μl of H₂SO₄ 2M. The plates were read with an automatic ELISA plate reader and the results were expressed as A=A_{450 nm}-A₆₂₀ₙₘ. The ELISA data were measured as the mean values of two independent assays.

Construction of λKM4

Plasmid pNS3785 (Hoess, 1995) was amplified by inverse PCR with the oligonucleotide sequences KT1 5′-TTTATCTAGACCCAGCCCTAGGAAGCTTCTCCTGAGTAGGACAAATCC-3′ bearing sites XbaI and AvrII (underlined) and KT2 5′-GGGTCTAGATAAAACGAAAGGCCCAGTCTTTC-3′ bearing XbaI for subsequent cloning in lambda phage. In the inverse PCR, a mixture of Taq polymerase and Pfu DNA polymerase was used to increase the fidelity of the DNA synthesis. Twenty-five amplification cycles were performed (95° C.-30 sec, 55° C.-30 sec, 72° C.-20 min). The self-ligation of the PCR product, previously digested with XbaI endonuclease, gave rise to plasmid pKM3. The lambda pD gene was amplified with PCR from plasmid pNS3785 using the primers K51 5′-CCGCCTTCCATGGGTACTAGTTTTAAATGCGGCCGCACGAGCAAAGAAACCTTTAC-3′ containing the restriction sites NcoI, SpeI, NotI (underlined) and K86 5′-CTCTCATCCGCCAAAACAGCC-3′. The PCR product was purified, digested with NcoI and EcoRI restriction endonucleases and re-cloned in the NcoI and EcoRI sites of pKM3, resulting in plasmid pKM4 bearing only the restriction sites SpeI and Not I at extremity 5′ of gpD. The plasmid was digested with XbaI enzyme and cloned in the XbaI site of lambda phage λDam15imm21nin5 (Hoess, 1995) (FIG. 1).

Construction of cDNA Libraries

mRNA was isolated from 10⁷ MCF-7 cells (T1 library) or from 0.1 g of a solid tumour sample (T4 library) using a QuickPrep Micro mRNA Purification Kit (Amersham Pharmacia Biotech) according to the manufacturer's instructions. Double-stranded cDNA was synthesised from 5 μg of poly(A)+ RNA using the TimeSaver cDNA Synthesis Kit (Amersham Pharmacia Biotech). Random tagged priming was performed as described previously (Santini, 1986). From 500 ng of double-stranded cDNA the first strand of cDNA copy was synthesised by using the random tagged primer 5′-GCGGCCGCTGG(N)₉-3′, and the second-strand cDNA copy by using the primer 5′-GGCGGCCAAC(N)₉-3′. The final cDNA product was amplified using oligonucleotides bearing SpeI with three different reading frames and NotI sites to facilitate cloning in the λKM4 lambda vector (5′-GCACTAGTGGCCGGCCAAC-3′,5′-GCACTAGTCGGCCGGCCAAC-3′,5′-GCACTAGTCGGGCCGGCCAAC-3′ and 5′-GGAGGCTCGAGCGGCCGCTGG-3′). The PCR products were purified on Quiaquick columns (Quiagen) and filtered on Microcon 100 (Amicon) to eliminate the small DNA fragments, digested with SpeI, NotI restriction enzymes, and, after extraction with phenol, filtered again on Microcon 100.

Vector λKM4 was digested with SpeI/NotI and dephosphorylated, and 8 ligation mixtures were prepared for each library, each containing 0.5 mg of vector and approximately 3 ng of insert. After overnight incubation at 4° C. the ligation mixtures were packaged in vitro with a lambda packaging kit (Ready-To-Go™ Lambda Packaging Kit, Amersham Pharmacia Biotech) and plated in top-agar on 100 (15 cm) NZY plates. After overnight incubation, the phage was eluted from the plates with SM buffer, purified, concentrated and stored at −80° C. in 7% DMSO SM buffer.

The complexity of the two libraries, calculated as total independent clones with inserts, was 108 for the T1 library and 3.6×10⁷ for the T4 library.

Selection by Affinity

For the identification of specific tumour antigens two different affinity selection procedures were used. The first consisted of two panning cycles with a positive serum (i.e. deriving from a patient suffering from tumour pathology), followed by an immunological screening procedure carried out with the same serum, or, alternatively, by analysis of clones taken at random from the mixture of selected phages. A second procedure used a mixture of sera from different patients for the selection, both for panning and for screening, for the purposes of increasing the efficacy of selection of cross-reactive antigens.

The T1 library was selected with 10 positive sera (B9, B11, B13, B14, B15, B16, B17, B18, B19, and B20), generating, after a single selection round, the corresponding pools p9^{I}, p11^{I}, p13^{I}, p14^{I}, p15^{I}, p16^{I}, p17^{I}, p18^{I}, p19^{I}, and p20^{I}. Each pool was then subjected to a second affinity selection round with the same serum, according to the first strategy mentioned above, generating a second series of pools (called p9^{II}, p11^{II}, p13^{II}, p14^{II}, p15^{II}, p16^{II}, p17^{II}, p18^{II}, p19^{II}, and p20^{II}). Some of the pools tested in ELISA demonstrated increased reactivity with the corresponding serum, thus confirming the efficacy of the library and of the affinity selection procedure. Individual clones from pools with increased reactivity (p9^{II}, p13^{II}, p15^{II}, p19^{II}, p20^{II}) were isolated by immunoscreening with sera used for the selection.

The second procedure mentioned above was applied to the p13^{II} pool, subjecting it to a third selection round with a mixture of sera with the exception of B13 (B11, B14, B15, B16, B17, B18, B19, and B20), and thus selecting cross-reactive clones. The resulting pool (p13^{III}) was assayed by ELISA with the same mixture of sera used in the panning. Individual clones from the pool were isolated by immunoscreening with mix ΔB13 (B11, B14, B15, B16, B17, B18, B19, and B20), which made it possible to isolate further positive clones.

Affinity selection experiments were also conducted with the T4 library (and also with the T1 library using different sera) according to the same methodology described here.

Multiple Immunological Screening (Pick-Blot Analysis)

The individual phage clones which were positive in the immunological screening were isolated and the eluted phages were grown on the lawn of bacteria on plates of 15 cm by picking in arrayed order. The plaques were transferred onto nitrocellulose membranes and subjected to analysis with different positive and negative sera. For the purposes of making the method more robust and reproducible, a Genesys Tekan robotic station was used to pick phages on the plates, which allowed analysis of up to a maximum of 396 individual clones on a membrane of 11×7.5 cm, or a lower number of clones repeatedly picked on the same plate cutting the membrane into smaller pieces before incubation with the sera.

Characterisation of Positive Clones

The clones that presented multiple reactivity, or a greater specificity for the sera of tumour patients as compared to that of healthy donors, were subsequently sequenced and compared with different databases of sequences currently available (Non-Redundant Genbank CDS, Non-Redundant Database of Genbank Est Division, Non-Redundant Genbank+EMBL+DDBJ+PDB Sequences).

The sequences obtained can be classified in six groups:
sequences that code for epitopes of known breast tumour antigens;
known sequences that code for epitopes of tumour antigens other than those of breast tumour;
sequences that code for autoantigens;
sequences that code for known proteins which are, however, not known to be involved either in tumours or in autoimmune diseases;
sequences that code for unknown proteins (e.g. EST);
new sequences not yet present in the databases.

Eighty-one different sequences were identified from the T1 library (called T1-1 to T1-115), 13% of which were unknown proteins and 16% were not present in the databases. Twenty-one sequences were identified from the T4 library (called T4-1 to T4-38), 40% of which were not to be found in the databases. The following table shows, by way of an example, the sequences of some of the clones selected:

| Name | | | |
|---|---|---|---|
| of | | Identi- | Classi- |
| clone | Sequence | fication | fication |
| T1-2 | ATGGGTACTAGTCGGCCGGCCAA | Intesti- | Tumor |
| | CATCACTCCCACCAATACAATGAC | nal mucin | antigen |
| | TTCTATGAGAACTACAACCTATTG | | |
| | GCCCACAGCCACAATGATGGAAC | | |
| | CACCTTCATCCACTGTATCAACTA | | |
| | CAGGCAGAGGTCAGACCACCTTT | | |
| | CCAGCTCTACAGCCACATTCCCC | | |
| | AATACCAAACACCCCAGCGGCCG | | |
| | C | | |
| T1-17 | ATGGGTACTAGTCGGGCCGGCCA | DNA-topo- | Tumor |
| | ACTTGTTGAAGAACTGGATAAAG | isomerase | antigen- |
| | TGGAATCTCAAGAACGAGAAGAT | II beta | malig- |
| | GTTCTGGCTGGAATGTCTGGAAA | | nant |
| | ATCCTCTTTCCAAAGATCTGAAGG | | meso- |
| | AGATTTTCTTTTAAGATCATTGAC | | thelioma |
| | CAGCGGCCGC | | |
| T1-8 | ATGGGTACTAGTGGCCGGCCAAC | RBP-1 | Tumor |
| | AAGGCAGCTGGAAGAGGTTCTCA | | antigen- |
| | AATTAGATCAAGAAATGCCTTTAA | | cancer |
| | CAGAAGTGAAGAGTGAACCTGAG | | of the |
| | GAAAATATCGATTCAAACAGTGA | | breast |
| | AAGTGAAAGAGAAGAGATAGAAT | | |
| | TAAAATCTCCGAGGGGACGAAGG | | |
| | AGAATTGCTCGAGATCCCAGCGG | | |
| | CCGC | | |
| T1-6 | ATGGGTACTAGTCGGGCCGGCCA | Golgin | Auto- |
| | ACTTGAGGAGCTGCAGAAGAAAT | p245 | antigen |
| | ACCAGCAAAAGCTAGAGCAGGAG | | |
| | GAGAACCCTGGCAATGATAATGT | | |
| | AACAATTATGGAGCTACAGACAC | | |
| | AGCTAGCACAGAAGACGACTTTA | | |
| | ATCAGTGATTCGAAATTGAAAGA | | |
| | GCAAGAGTTCAGAGAACAGATTC | | |
| | ACAATTTAGAAGACCGTTTGAAG | | |
| | AAATATGAAAAGAATGTATATGC | | |
| | AACAACTGTGGGGACACCTTACA | | |
| | AAGGTGGCAATTTGTACCATACG | | |
| | GATGTCTCACTCTTTGGAGAACCT | | |
| | ACCAGCGGCCGC | | |
| T1-101 | ATGGGTACTAGTCGGCCGGCCAA | Human | Auto- |
| | CTTCGTGGAAATCAGTGAAGATA | lupus La | antigen |
| | AAACTAAAATCAGAAGGTCTCCA | protein | |
| | AGCAAACCCCTACCTGAAGTGAC | | |
| | TGATGAGTATAAAAATGATGTAA | | |
| | AAAACAGATCTGTTTATATTAAAG | | |
| | GCTTCCCAACTGAAGCCAGCGGC | | |
| | CGC | | |
| T1-52 | GTGGCCGGCCAACGTTATCAGAG | Binding | Unknown |
| | TAGAAGTGGGCATGATCAGAAGA | protein | as tumor |
| | ATCATAGAAAGCATCATGGGAAG | p53 | antigen |
| | AAAAGAATGAAAAGTAAACGATC | | |
| | TACATCATTGTCATCTCCCAGAAA | | |
| | CGGAACCAGCGGCCGC | | |
| T1-35 | ATGGGTACTAGTCGGGCCGGCCA | Nuclear | Unknown |
| | ACAAATTAGGCAGATTGAGTGTG | matrix | as tumor |
| | ACAGTGAAGACATGAAGATGAGA | protein | antigen |
| | GCTAAGCAGCTCCTGGTTGCCTG | | |
| | GCAAGATCAAGAGGGAGTTCATG | | |
| | CAACACCTGAGAATCTGATTAAT | | |
| | GCACTGAATAAGTCTGGATTAAG | | |
| | TGACCTTGCAGAAAGTCCCAGCG | | |
| | GCCGC | | |
| T1-10 | ATGGGTACTAGTGGCCGGCCAAC | Ribosomal | Unknown |
| | GGCAGTAGTTCTGGAAAAGCCAC | protein | as tumor |
| | TGGGGACGAGACAGGTGCTAAAG | s3a | antigen |
| | TTGAACGAGCTGATGGAGCTTCA | | |
| | TGGTGAAGGCAGTAGTTCTGGAA | | |
| | AAGCCACTGGGGACGAGACAGGT | | |
| | GCTAAAGTTGAACGAGCTGATGG | | |
| | AATGACCCCCAGCGGCCGC | | |
| T1-39 | ATGGGTACTAGTGGCCGGCCAAC | No data | |
| | GAATTATTCGAGTGCTATAGGCG | | |
| | CTTGTCAGGGAGGTAGCGATGAG | | |
| | AGTAATAGATAGGGCTCAGGCGT | | |
| | TTGTTGATGAGATATTTGGAGGT | | |
| | GGGGATGATGCACATAATTTGAA | | |
| | TCAACACAACTCCAGCGGCCGC | | |
| T1-12 | ATGGGTACTAGTCGGGCCGGCCA | No data | |
| | ACGTGGTATTATTTAAAAATAGCT | | |
| | AAAAAGGTAAACAATCCAAATGC | | |
| | CATTAAACAGAGAATTTTAAAAAA | | |
| | TGAGATACTACACAGCAACAAAA | | |
| | ACCTATGAGCTAATGCTAGATGC | | |
| | AACAACACAGACCAGCGGCCGC | | |
| T1-32 | ATGGGTACTAGTCGGGCCGGCCA | No data | |
| | ACTACACGCCTTTCCACTC | | |
| | CACTCTACTACACTCTACTACACT | | |
| | ACACCCAGCGGCCGC | | |
| T1-74 | ATGGGTACTAGTCGGCCGGCCAA | EST | |
| | CAGAGAAGCTAAGCAACTGCATC | | |
| | ATCAGCCACATTCAATCGAATTAA | | |
| | TACAGTCCAGCGGCCGC | | |
| T4-2 | ATGGGTACTAGTCGGCCGGCCAA | EST | |
| | CTCAGAGGTGTATAAGCCAACAT | | |
| | TGCTCTACTCCAGCGGCCGC | | |
| T4-11 | ATGGGTACTAGTGGCCGGCCAAC | EST | |
| | GGTTGGTTTTACTCTAGATTTCAC | | |
| | TGTCGACCCACCCAGCGGCCGC | | |
| T4-19 | ATGGGTACTAGTCGGGCCGGCCA | No data | |
| | ACTATACCGTACAACCCTAACATA | | |
| | TACCAGCGGCCGC | | |
| T5-8 | ATGGGTACTAGTCGGGCCGGCCA | AKAP | Unknown |
| | ACAGAGAGAGCAAGAAAAGAAAA | protein | as |
| | GAAGCCCTCAAGATGTTGAAGTTC | | tumour |
| | TCAAGACAACTACTGAGCTATTTC | | antigen |
| | ATAGCAATGAAGAAAGTGGATTTT | | |
| | TTAATGAACTCGAGGCTCTTAGAG | | |
| | CTGAATCAGTGGCTACCAAAGCA | | |
| | GAACTTGCCAGTTATAAAGAAAAG | | |
| | GCTGAAAAACTTCAAGAAGAACTT | | |
| | TTGGTAAAAGAAACAAATATGACA | | |
| | TCTCTTCAGAAAGACTTAAGCCAA | | |
| | GTTAGGGATCACCAGGGCCGC | | |
| T5-13 | ATGGGTACTAGTCGGGCCGGCCA | SOS1 | Unknown |
| | ACACGCATTCGAGCAAATACCAA | protein | as |
| | GTCGCCAGAAGAAAATTTTAGAA | | tumour |
| | GAAGCTCATGAATTGAGTGAAGA | | antigen |
| | TCACTATAAGAAATATTTGGCAAA | | |
| | ACTCAGGTCTATTAATCCACCATG | | |
| | TGTGCCTTTCTTTGGAATTTATCT | | |
| | CACTAATCTCTTGAAAACAGAAGA | | |
| | AGGCAACCCTGAGGTCCTAAAAA | | |
| | GACATGGAAAAGAGCTTATAAACT | | |
| | TTAGCAAAAGGAGGAAAGTAGCA | | |
| | GAAATAACAGGAGAGATCCAGCA | | |
| | GTACCAAAATCAGCCNTACTGTTT | | |
| | ACGAGTAGAATCAGATATCAAAA | | |
| | GGTTCTTTGAAAACTTGAATCCGA | | |
| | TGGGAAATAGCATGGAGAAGGAA | | |
| | TTTACAGATTATCTTTTCAACAAA | | |
| | TCCCTAGAAATAGAACCACGAAAA | | |
| | CCCAGCGGCCGC | | |
| T5-15 | ATGGGTACTAGTCGGGCCGGCCA | EST | |
| | ACAGGAGAGGTCCTTGGCCCTCT | KIAA1735 | |
| | GTGAACCAGGTGTCAATCCCGAG | protein | |
| | GAACAACTGATTATAATCCAAAGT | | |
| | CGTCTGGATCAGAGTTTGGAGGA | | |
| | GAATCAGGACTTAAAGAAGGAAC | | |
| | TGCTGAAATGTAAACAAGAAGCC | | |
| | AGAAACTTACAGGGGATAAAGGA | | |
| | TGCCTTGCAGCAGAGATTGACTCA | | |
| | GCAGGACACATCTGTTCTTCAGCT | | |
| | CAAACAAGAGCTACTGAGGGCAA | | |
| | ATATGGACAAAGATGAGCTGCAC | | |
| | AACCAGAATGTGGATCTGCAGAG | | |
| | GAAGCTAGATGAGAGGACCCAGC | | |
| | GGCCGC | | |
| T5-18 | ATGGGTACTAGTCGGGCCGGCCA | mic onco- | Unknown |
| | ACCGATGTCTGGACATGGGAGTT | gen, al- | as |
| | TTCAAGAGGTGCCACGTCTCCACA | ternative | tumour |
| | CATCAGCACAACTACGCAGCGCC | frame | antigen |
| | TCCCTCCACTCGGAAGGACTATCC | | |
| | TGCTGCCAAGAGGGTCAAGTTGG | | |
| | ACAGTGTCAGAGTCCTGAGACAG | | |
| | ATCAGCAACAACCGAAAATGCAC | | |
| | CAACCCAGCGGCCGC | | |
| T6-1 | ACTAGTCGGGCCGGCCAACGTTAT | protein | known as |
| | GAGAAGTCAGATAGTAGCGATAGT | kinase C- | cuta- |
| | GAGTATATCAGTGATGATGAGCAG | binding | neous T- |
| | AAGTCTAAGAACGAGCCAGAAGAC | protein | cell |
| | ACAGAGGACAAAGAAGGTTGTCAG | | lymphoma |
| | ATGGACAAAGAGCCATCTGCTGTT | | tumor |
| | AAAAAAAAGCCCAAGCCTACAAAC | | antigen |
| | CCAGTGGAGATTAAAGAGGAGCTT | | |
| | AAAAGCACGCCACCAGCCAGCGG | | |
| | CCGC | | |
| T6-2 | ACTAGTCGGGCCGGCCAACTTGCC | not found | |
| | AGGATTCCCTCAGTAACGGCGAGT | | |
| | GAACAGGGAAGAACCAGCGGCCG | | |
| | C | | |
| T6-6 | ACTAGTGGGCCGGCCAACGCTGCT | homolo- | Unknown |
| | CCACCCTCAGCAGATGATAATATC | gous to | as |
| | AAGACACCTGCCGAGCGTCTGCGG | PI-3-ki- | tumour |
| | GGGCCGCTTCCACCCTCAGCGGAT | nase re- | antigen |
| | GATAATCTCAAGACACCTTCCGAG | lated | |
| | CGTCAGCTCACTCCCCTCCCCCCA | kinase | |
| | GCGGCCGC | SMG-1 | |
| T6-7 | ACTAGTCGGGCCGGCCAACGGGA | Fucosyl- | Unknown |
| | ATTGGGAAGGACGGGCCTATATCC | transfer- | as |
| | CTCCTACAAAGTTCGAGAGAAGAT | ase | tumour |
| | AGAAACGGTCAAGTACCCCACATA | | antigen |
| | TCCTGAGGCTGAGAAATAAAGCTC | | |
| | AGATGGAAGAGATAAACGACCAAA | | |
| | CTCAGTTCGACCAAACTCAGTTCA | | |
| | AACCATTTGAGCCAAACTGTAGAT | | |
| | GAAGAGGGCTCTGATCTAACAAAA | | |
| | TAAGGTTATATGAGTAGATACTCT | | |
| | CAGCACCAAGAGCAGCTGGGAACT | | |
| | GACATAGGCTTCAATTGGTGGAAT | | |
| | TCCTCTTTAACAAGGGCTGCAATG | | |
| | CCCTCATACCCATGCACAGTACAA | | |
| | TAATGTACTCACATATAACATGCA | | |
| | AAGGTTGTTTTCTACTTTGCCCCTT | | |
| | TCAGTATGTCCCCATAAGACAAAC | | |
| | ACTACCAGCGGCCGC | | |
| T7-1 | ACTAGTGTCCTGGAACCCACAAAA | EST | Unknown |
| | GTAACCTTTTCTGTTTCACCGATT | KIAA1288 | as |
| | GAAGCGACGGAGAAATGTAAGAA | protein | tumour |
| | AGTGGAGAAGGGTAATCGAGGGC | | antigen |
| | TTAAAAACATACCAGACTCGAAGG | | |
| | AGGCACCTGTGAACCTGTGTAAAC | | |
| | CTAGTTTAGGAAAATCAACAATCA | | |
| | AAACGAATACCCCAATAGGCTGCA | | |
| | AAGTTAGAAAAACTGAAATTATAA | | |
| | GTTACCCAAGTACCAGCGGCCGC | | |
| T9-22 | ATGGACTTAACAGCTGTTTACAGA | similar | |
| | ACATTCCACCCAACAATCACAGAA | to re- | |
| | TATACATTCTATTTAACAGTGCAT | verse | |
| | GGAACTTTTTCCAAGATAGACCAT | trascrip- | |
| | ATGATAGGCCACAAAACAAGTCTC | tase | |
| | AATAAGTCTAAGAAAACTGAAATT | homolog, | |
| | ATATCAAGTACTCTCTCAGACCAC | 50% of | |
| | AGTGGAATAAAATTGGAAAGTAAT | identity | |
| | TCCAAAAGGAACCCCCAAATCCAT | | |
| | GCCAGCGGCCGC | | |
| T11-5 | ATGCCGATTGACGTTGTTTACACC | EST | |
| | TGGGTGAATGGCACAGATCTTGAA | unnamed | |
| | CTACTGAAGGAACTACAGCAGGTC | trans- | |
| | AGAGAACAGATGGAGGAGGAGCA | membrane | |
| | GAAAGCAATGAGAGAAATCCTTGG | protein | |
| | GAAAAACACAACGGAACCTACTAA | | |
| | GAAGAGGTCCTACTTTGTGAATTT | | |
| | TCTAGCCGTGTCCAGCGGCCGC | | |
| T11-6 | ACTAGTGGCCGGCCAACGTATAA | zinc | Unknown |
| | AGTAAATATTTCTAAAGCAAAAA | finger | as |
| | CTGCTGTGACGGAGCTCCCTTCT | protein | tumour |
| | GCAAGGACAGATACAACACCAGT | 258 | antigen |
| | TATAACCAGTGTGATGTCATTGG | | |
| | CAAAAATACCTGCTACCTTATCT | | |
| | ACAGGGAACACTAACAGTGTTTT | | |
| | AAAAGGTGCAGTTACTAAAGAGG | | |
| | CAGCAAAGATCATTCAAGATGAA | | |
| | AGTACACAGGAAGATGCTATGAA | | |
| | ATTTCCATCTTCCCAATCTTCCCA | | |
| | GCCTTCCAGGCTTTTAAAGAACA | | |
| | AAGGCATATCATGCAAACCGGTC | | |
| | ACACATCCCAGCGGCCGC | | |
| T11-9 | ACTAGTCGGGCCGGCCAACTTCG | EST | |
| | ATTTAGTGATCATGCCGTGTTGA | hypoteti- | |
| | AATCCTTGTCTCCTGTAGACCCA | cal human | |
| | GTGGAACCCATAAGTAATTCAGA | protein | |
| | ACCATCAATGAATTCAGATATGG | | |
| | GAAAAGTCAGTAAAAATGATACT | | |
| | GAAGAGGAAAGTAATAAATCCGC | | |
| | CACAACAGACAATGAAATAAGTA | | |
| | GGACTGAGTATTTATGTGAAAAC | | |
| | TCTCTAGAAGGTAAAAATAAAGA | | |
| | TAATTCTTCAAATGAAGTCTTCC | | |
| | CCCAATATGCCAGCGGCCGC | | |
| T11-3 | ACTAGTCGGGCCGGCCAACGCAA | EST | |
| | GCAAAGTTTCCCAAATTCAGATC | KIAA0697 | |
| | CTTTACATCAGTCTGATACTTCC | protein | |
| | AAAGCTCCAGGTTTTAGACCACC | | |
| | ATTACAGAGACCTGCTCCAAGTC | | |
| | CCTCAGGTATTGTCAATATGGAC | | |
| | TCGCCATATGGTTCTGTAACACC | | |
| | TTCTTCAACACATTTGGGAAACT | | |
| | TTGCTTCAAACATTTCAGGAGGT | | |
| | CAGATGTACGGACCTGGGGCACC | | |
| | CCTTGGAGGAGCACCCACCAGCG | | |
| | GCCGC | | |
| T5-2 | ATGGGTACTAGTCGGGCCGGCCA | human | |
| | ACCCACTTCAGAAAACTATTTGG | genome | |
| | CAGTAACTACTAAAACTAAACAT | DNA | |
| | AAGCATAGCCTACAACCCAGTAA | | |
| | TGCCAGTATTTCACTCCTAGGTA | | |
| | TATACCCAACCCCCAGCGGCCGC | | |
| T5-19 | ACTAGTCGGGCCGGCCAACGTGA | EST | |
| | CACACAGACACATGCACATGTGA | | |
| | GTGTATGCGTGCACACACCCCAC | | |
| | CACACCTACAAATACCCCACCAG | | |
| | CGGCCGC | | |

Clone T1-52 is known as a fragment of binding protein p53 (Haluska P. et al., NAR, 1999, v. 27, n. 12, 2538-2544), but has never been identified as a tumour antigen. Said clone has the sequence VLVAGQRYQSRSGHDQKNHRKHHGKKRMKSKRSTSLSSPRNGTSGR and its use as a tumour antigen is part of the invention described herein.

Clone T1-17 is known as a fragment of DNA-topoisomerase II beta identified as malignant mesothelioma tumour antigen (Robinson C., et al. Am. J. Respir. Cell. Mol. Biol. 2000;22:550-56). The present invention has identified it as breast cancer tumour antigen. Said clone has the sequence MGTSRAGQLVEELDKVESQEREDVLAGMSGKSSFQRSEGDFLLRSLTSGR and it use as a breast cancer tumour antigen is part of the invention described herein.

Clone T1-32, hitherto unknown, has the following sequence MGTSRAGQLHAFPLHSTTLYYTTPSGR; it is a tumour antigen and as such is part of the invention described herein.

Clone T1-74, hitherto unknown, has the following sequence MGTSRPANREAKQLHHQPHSIELIQSSGR; it is a tumour antigen and as such is part of the invention described herein.

Clone T4-2, hitherto unknown, has the following sequence MGTSRPANSEVYIKPTLLYSSGR; it is a tumour antigen and as such is part of the invention described herein.

Clone T4-11, hitherto unknown, has the following sequence MGTSGRPTVGFTLDFTVDPPSGR; it is a tumour antigen and as such is part of the invention described herein.

Clone T4-19, hitherto unknown has the following sequence MGTSRAGQLYRTTLTYTSGR; it is a tumour antigen and as such is part of the invention described herein.

Clone T1-12, hitherto unknown, has the following sequence MRYYTATKTYELMLDATTQTSGR; it is a tumour antigen and as such is part of the invention described herein.

Clone T1-39, hitherto unknown, has the following sequence MRVIDRAQAFVDEIFGGGDDAHNLNQHNSSGR; it is a tumour antigen and as such is part of the invention described herein.

Clone T5-8 is known as a fragment of AKAP protein, but has never been identified as a tumour antigen. Said clone has the sequence MGTSRAGQQREQEKKRSPQDVEVLKTTTELFHSNEESGFFNELEALRAESVATKAELASYKEKAEKLQEELLVKETNMTSLQKDLSQVRDHQGRG and its use as a tumour antigen is part of the invention described herein.

Clone T5-13 is known as as a fragment of SOS1 protein, but has never been identified as a tumour antigen. Said clone has the sequence AGTSRAGQHAFEQIPSRQKKILEEAHELSEDHYKKYRSINPPCVPFFGIYLTNLLKTEEGNPEVLKRHGIKLINFSKRRKVAEITGEIQQYQNQYCLRVESDIKRFFENLNPMGNSMEKEFTDYLFNKSLEIEPRKPSGR and its use as a tumour antigen is part of the invention described herein.

Clone T5-15 is known as a fragment of EST protein KIAA1735, but has never been identified as a tumour antigen. Said clone has the sequence MGTSRAGQQERSLALCEPGVNPEEQLIIIQSRLDQSLEENQDLKKELLKCKQEARNLQGIKDALQQRLTQQDTSVLQLKQELLRANMDKDELHNQNVDLQRKLDERTQRP and its use as a tumour antigen is part of the invention described herein.

Clone T5-18 is known as as a fragment of a mic oncogen, alternative frame, but has never been identified as a tumour antigen. Said clone has the sequence MGTSRAGQPMSGHGSFQEVPRLHTSAQLRSASLHSEGLSCCQEGQVGQCQSPETDQQQPKMHQPSGR and its use as a tumour antigen is part of the invention described herein.

Clone T6-1 is known as a fragment of protein kinase C-binding protein, identified as cutaneous T-cell lymphoma tumour antigen (Eichmuller S., et al. PNAS, 2001; 98; 629-34). The present invention has identified it as breast cancer tumour antigen. Said clone has the sequence TSRAGQRYEKSDSSDSEYISDDEQKSKNEPEDTEDKEGCQMDKEPSAVKKKPKPTNPVEIKEELKSTPPA and its use as a breast cancer tumour antigen is part of the invention described herein.

Clone T6-2 hitherto unknown, has the following sequence TSRAGQLARIPSVTASEQGRT; it is a tumour antigen and as such is part of the invention described herein.

Clone T6-6 is known as a fragment of homologous to PI-3-kinase related kinase SMG-1, but has never been identified as a tumour antigen. Said clone has the sequence TSGPANAAPPSADDNIKTPAERLRGPLPPSADDNLKTPSERQLTPLPPAAAK; it is a tumour antigen and as such is part of the invention described herein.

Clone T6-7 is known as a fragment of fucosyltransferase, but has never been identified as a tumour antigen. Said clone has the sequence TSRAGQRELGRTGLYPSYITREICETVKYPTYPEAEK; it is a tumour antigen and as such is part of the invention described herein.

Clone T7-1 is known as a fragment of EST protein KIAA1288, but has never been identified as a tumour antigen. Said clone has the sequence TSVLEPTKVTFSVSPIEATEKCKKVEKGNRGLKNIPDSKEAPVNLCKPSLGKSTIKTNTPIGCKVRKTEIISYPSTSGR; it is a tumour antigen and as such is part of the invention described herein.

Clone T9-22 is known as a fragment of similar (50% of identity) to reverse trascriptase homolog protein, but has never been identified as a tumour antigen. Said clone has the sequence MDLTAVYRTFHPTITEYTFYLTVHGTFSKIDHMIGHKTSLNKSKKTEIISSTLSDHSGIKLESNSKRNPQIHASGR; it is a tumour antigen and as such is part of the invention described herein.

Clone T11-5 is known as a fragment of an unnamed transmembrane theoretical protein, but has never been identified as a tumour antigen. Said clone has the sequence MPIDVVYTWVNGTDLELLKELQQVREQMEEEQKAMREILGKNTTEPTKKRSYFVNFLAVSSGR; it is a tumour antigen and as such is part of the invention described herein.

Clone T11-6 is known as a fragment of the zinc finger protein 258, but has never been identified as a tumour antigen. Said clone has the sequence TSGRPTYKVNISKAKTAVTELPSARTDTTPVITSVMSLAKIPATLSTGNTNSVLKGAVTKEAAKIIQDESTQEDAMKFPSSQSSQPSRLLKNKGISCKPVTHPSGR; it is a tumour antigen and as such is part of the invention described herein.

Clone T11-9 is known as a fragment of a hypotetical human protein, but has never been identified as a tumour antigen. Said clone has the sequence TSRAGQLRFSDHIAVLKSLSPVDPVEPISNSEPSMNSDMGKVSKNDTEEESNKSATTDNEISRTEYLCENSLEGKNKDNSSNEVFPQYASGR; it is a tumour antigen and as such is part of the invention described herein.

Clone T11-3 is known as a fragment of EST protein KIAA0697, but has never been identified as a tumour antigen. Said clone has the sequence TSRAGQRKQSFPNSDPLHQSDTSKAPGFRPPLQRPAPSPSGIVNMDSPYGSVTPSSTHLGNFASNISGGQMYGPGAPLGGAPTSGR; it is a tumour antigen and as such is part of the invention described herein.

Clone T5-2 is known as a fragment of human genome DNA, but has never been identified as a tumour antigen. Said clone has the sequence MGTSRAGQPTSENYLAVTTKTKHKHSLQPSNASISLLGIYPTPSGR; it is a tumour antigen and as such is part of the invention described herein.

Clone T5-19 is known as a fragment of EST protein, but has never been identified as a tumour antigen. Said clone has the sequence TSRAGQRDTQTHAHVSVCVHTPHHTYKYPTSGR; it is a tumour antigen and as such is part of the invention described herein.

It will be understood that, according to the present invention, sequences which are part of known proteins but were unknown as tumor antigen are an object of the present invention as far as their use as tumor antigens is concerned. In the same way, an object of the present invention are the use as tumour antigen of the sequence, or of the entire or part of the product of the gene encoding for said sequence.

The phage clones characterised by means of pick-blot analysis and for which specific reactivity had been demonstrated with sera from patients suffering from breast tumours were amplified and then analysed with a large panel of positive and negative sera. After this ELISA study, the cDNA clones regarded as corresponding to specific tumour antigens were cloned in different bacterial expression systems (protein D and/or GST), for the purposes of better determining their specificity and selectivity. To produce the fusion proteins each clone was amplified from a single plaque by PCR using the following oligonucleotides: K84 5′-CGATTAAATAAGGAGGAATAAACC-3′ and K86 5′-CTCTCATCCGCCAAACAGCC-3′. The resulting fragment was then purified using the QIAGEN Purification Kit, digested with the restriction enzymes SpeI and NotI and cloned in plasmid pKM4-6H to produce the fusion protein with D having a 6-histidine tail, or in vector pGEX-SN to generate the fusion with GST. The corresponding recombinant proteins were then prepared and purified by means of standard protocols (Sambrook, J., Fritsch, E. F. & Maniatis, T. (1989) Molecular Cloning, Cold Spring Harbor Laboratory Press, Cold Spring Harbor).

The following table gives, by way of an example, the reactivities with negative and positive sera of a number of selected clones, assayed in the form of phage or fusion protein preparations:

| | Lambda phage | Lambda | Reactivity | Reactivity of |
|---|---|---|---|---|
| | reactivity with | phage | of fusion | fusion |
| | positive sera | reactivity | protein D | protein D |
| | (number positive/ | with | with positive | with negative |
| Name | total number | negative | sera (* for GST | sera (* for |
| of clone | assayed) | sera | fusion) | GST fusion) |
| T1-2 | 1/20 | 0/9 | | |
| T1-17 | 1/10 | 0/0 | * 2/16 | * 0/15 |
| T1-8 | 1/10 | 0/0 | 1/13 | 0/15 |
| T1-6 | 1/10 | 0/0 | | |
| T1-101 | /20 | 0/1 | | |
| T1-52 | 7/41 | 0/20 | 13/53 | 3/24 |
| T1-35 | 4/10 | 14/21 | | |
| T1-10 | 1/10 | 0/0 | | |
| T1-39 | 11/34 | 0/26 | Non-reactive | |
| T1-12 | 23/72 | 0/31 | Non-reactive | |
| T1-32 | 17/72 | 0/31 | * 10/72 | * 1/31 |
| T1-74 | 29/72 | 2/27 | * 21/72 | * 4/32 |
| T4-2 | 11/18 | 0/17 | 9/28 | 1/31 |
| T4-11 | 4/21 | 0/26 | 8/70 | 0/30 |
| T4-19 | 5/20 | 0/26 | 12/70 | 0/30 |

For the purposes of demonstrating the efficacy of the tumour antigens selected for recognising tumour cells and thus for the detection and diagnosis of pathological abnormalities, mice were immunised to induce an antibody response to a number of the clones selected.

The mice were immunised by giving seven administrations of the antigen over a period of two months, using as immunogens the fusion proteins D1-52, D4-11 and D4-19, corresponding to the fusions of the sequences of clones T1-52, T4-11 and T4-19 with protein D. Each time, 20 μg of protein were injected (intraperitoneally or subcutaneously) per mouse in CFA, 20 μg in IFA, 10 μg in PBS and four times 5 μg in PBS for each of the three proteins. For the purposes of checking the efficacy of immunisation to the sequence of the tumour antigen, the sera of the immunised animals were assayed against the same peptide sequences cloned in different contexts, in order to rule out reactivity to protein D.

In the case of D1-52, the sera of the immunised mice were assayed with the fusions with GST (GST1-52), whereas in the cases of D4-11 and D4-19 the corresponding peptide sequences were cloned in vector pC89 (Felici et al. 1991, J. Mol. Biol. 222:301-310) and then tested as fusions to pVIII (major coat protein of filamentous bacteriophages). The results of ELISA with the sera of the immunised animals showed that effective immunisation was obtained in the cases of D1-52 and D4-11, and thus the corresponding sera were assayed for the ability to recognise tumour cells. To this end, the cell line MCF7 was used, and analysis by FACS demonstrated that antibodies present in both sera (anti-D1-52 and anti-D4-11) are capable of specifically recognising breast tumour MCF7 cells, and not, for instance, ovarian tumour cells, while this recognition capability is not present in preimmune sera from the same mice.

1. Specific tumor antigens obtainable by selection of cDNA libraries with sera, characterised in that said selection is accomplished with the phage display technique.

2. Tumor antigens according to claim 1, in which said selection is accomplished by means of the SEREX technique (serological analysis of autologous tumor antigens through expression of recombinant cDNA).

3. Tumor antigens according to claim 1, in which said selection is accomplished by means of the affinity selection technique.

4. Tumor antigens according to claim 1, in which said libraries are obtained from tumor biopsies.

5. Tumor antigens according to claim 1, in which said libraries are obtained from cultured tumor cell lines.

6. Antigen according to claim 6 selected from the group consiting of:

| | |
|---|---|
| MGTSRPANREAKQLHHQPHSIELIQSSGR; | (SEQ ID NO: 49) |
| MGTSRPANSEVYKPTLLYSSGR; | (SEQ ID NO: 50) |
| MGTSGRPTVGFTLDFTVDPPSGR; | (SEQ ID NO: 51) |
| MGTSRAGQLYRTTLTYTSGR; | (SEQ ID NO: 52) |
| MGTSRAGQLHAFPLHSTTLYYTTPSGR; | (SEQ ID NO: 48) |
| MRYYTATKTYELMLDATTQTSGR; | (SEQ ID NO: 53) |
| MRVIDRAQAFVDEIFGGGDDAHNLNQHNSSGR. | (SEQ ID NO: 54) |

7. Use as tumor antigen of the sequence or of the entire or part of the product of the gene encoding for said sequence selected from the group consisting of:

| | |
|---|---|
| VLVAGQRYQSRSGHDQKNHRKHHGKKRMKSKRSTSLSSPRNGT-SGR; | (SEQ ID NO: 46) |
| MGTSRAGQQREQEKKRSPQDVEVLKTTTELFHSNEESGFFNELE- | (SEQ ID NO: 55) |
| ALRAESVATKAELASYKEKAEKLQEELLVKETNMTSLQKDLSQVRDHQGRG; | |
| AGTSRAGQHAFEQIPSRQKKILEEAHELSEDHYKKYLAKLRSINP- | (SEQ ID NO: 56) |
| PCVPFFGIYLTNLLKTEEGNPEVLKRHGKELINFSKRRKVAEITGEIQQYQNQYC | |
| LRVESDIKRFFENLNPMGNSMEKEFTDYLFNKSLEIEPRKPSGR; | |
| MGTSRAGQQERSLALCEPGVNPEEQLIIIQSRLDQSLEENQDLKK- | (SEQ ID NO: 57) |
| ELLKCKQEARNLQGJKDALQQRLTQQDTSVLQLKQELLRANMDKDELHNQNV | |
| DLQRKLDERTQRP; | |
| MGTSRAGQPMSGHGSFQEVPRLHTSAQLRSASLHSEGLSCCQEG- | (SEQ ID NO: 58) |
| QVGQCQSPETDQQQPKMHQPSGR; | |
| TSRAGQLARIPSVTASEQGRT; | (SEQ ID NO: 60) |
| TSGPANAAPPSADDNIKTPAERLRGPLPPSADDNLKTPSERQLTP- | (SEQ ID NO: 61) |
| LPPAAAK; | |
| TSRAGQRELGRTGLYPSYKVREKIETVKYPTYPEAEK; | (SEQ ID NO: 62) |
| TSVLEPTKVTFSVSPIEATEKCKKVEKGNRGLKNIPDSKEAPVNL- | (SEQ ID NO: 63) |
| CKPSLGKSTIKTNTPIGCKVRKTEIISYPSTSGR; | |
| MDLTAVYRTFHPTITEYTFYLTVHGTFSKIDHMIGHKTSLNKSKK- | (SEQ ID NO: 64) |
| TEIISSTLSDHSGIKLESNSKRNPQIHASGR; | |
| MPIDVVYTWVNGTDLELLKELQQVREQMEEEQKAMREILGKNT- | (SEQ ID NO: 65) |
| TEPTKKRSYFVNFLAVSSGR; | |
| TSGRPTYKVNISKAKTAVTELPSARTDTTPVITSVMSLAKIPATLST- | (SEQ ID NO: 66) |
| GNTNSVLKGAVTKEAAKIIQDESTQEDAMKFPSSQSSQPSRLLKNKGISCKPVT | |
| HPSGR; | |
| TSRAGQLRFSDHAVLKSLSPVDPVEPISNSEPSMNSDMGKVSKN- | (SEQ ID NO: 67) |
| DTEEESNKSATTDNEISRTEYLCENSLEGKNKDNSSNEVFPQYASGR; | |
| TSRAGQRKQSFPNSDPLHQSDTSKAPGFRPPLQRPAPSPSGIVNM- | (SEQ ID NO: 68) |
| DSPYGSVTPSSTHLGNFASNISGGQMYGPGAPLGGAPTSGR; | |
| MGTSRAGQPTSENYLAVTTKTKHKHSLQPSNASISLLGIYPTPSGR; | (SEQ ID NO: 69) |
| TSRAGQRDTQTHAHVSVCVHTPHHTYKYPTSGR. | (SEQ ID NO: 70) |

8. Use of the antigen or of the entire or part of the product of the gene encoding for said sequence selected from the group consisting of:
(SEQ ID NO: 59)
TSRAGQRYEKSDSSDSEYISDDEQKSKNEPEDTEDKEGCQMDKE-
PSAVKKKPKPTNPVEIKEELKSTPPA;
(SEQ ID NO: 47)
MGTSRAGQLVEELDKVESQEREDVLAGMSGKSSFQRSEGDFLLR-
SLTSGR
as a breast cancer tumour antigen.

9. Use of antigens of claim 1 as active agents useful for the preparation of medicaments for the treatment of tumors.

10. Specific ligand for an antigen of claim 1.

11. Anti-antigen antibody of claim 1.

12. Use of a ligand of claim 10 or of an antibody of claim 11 as active agent for the preparation of medicaments for the treatment of tumors.

13. Use of a ligand of claim 10 or of an antibody of claim 11 as carrier for an active agent for the treatment of tumors.

14. Use of a ligand of claim 12 or of an antibody of claim 13 for the preparation of target-specific contrast media.

15. Use of the expression/display vector (λKM4) for obtaining antigens of claim 1.

16. Antitumor vaccine comprising at least an antigen of claim 1.

17. Antitumor medicament comprising a ligand of claim 10.

18. Antitumor medicament comprising an antibody of claim 10.

19. Vaccine for treating breast cancer comprising the antigen of claim 8 and/or a specific ligand thereof and/or a specific antibody thereof.
